(19) United States
(12) Patent Application Publication
(10) Pub. No.: US 2025/0280485 A1
(43) Pub. Date: Sep. 4, 2025

(54) METHOD FOR CONTROLLING THE TEMPERATURE OF AN X-RAY DEVICE, X-RAY DEVICE AND COMPUTER PROGRAM PRODUCT

(71) Applicant: Siemens Healthineers AG, Forchheim (DE)

(72) Inventors: Bjoern KREISLER, Hausen (DE); Andreas KRETSCHMANN, Langensendelbach (DE); Stefan WOELFEL, Dormitz (DE); Thomas HOFMANN, Grossenseebach (DE); Bodo REITZ, Forchheim (DE)

(73) Assignee: Siemens Healthineers AG, Forchheim (DE)

(21) Appl. No.: 19/063,507

(22) Filed: Feb. 26, 2025

(30) Foreign Application Priority Data
Feb. 29, 2024 (DE) 10 2024 201 890.2

(51) Int. Cl. H05G 1/02; H05G 1/30

(52) U.S. Cl. CPC H05G 1/025; H05G 1/30

(57) ABSTRACT

A method for controlling a temperature of an X-ray device, comprises: acquiring planning information, including at least one operating parameter of at least one component of the X-ray device, for a planned operation of the X-ray device; identifying a planning temperature of the at least one component of the X-ray device based on the at least one operating parameter; operating the X-ray device in accordance with the planning information; and controlling a temperature control unit of the X-ray device prior to and/or during operation of the X-ray device based on the planning temperature such that the temperature control unit controls a temperature of the at least one component of the X-ray device to a defined temperature or a defined temperature range by providing a heating capacity and/or a cooling capacity.

### CROSS-REFERENCE TO RELATED APPLICATION(S)

The present application claims priority under 35 U.S.C. § 119 to German Patent Application No. 10 2024 201 890.2, filed Feb. 29, 2024, the entire contents of which is incorporated herein by reference.

### FIELD

One or more embodiments of the present invention relate to a method for controlling the temperature of an X-ray device, an X-ray device and a computer program product.

### BACKGROUND

Modern computer tomography devices (CT devices) or computed tomography devices have a gantry with a rotatable frame, on which, inter alia, the X-ray source and an X-ray detector for detecting X-rays are arranged. An X-ray detector of this type usually comprises an X-ray converter element which has an X-ray sensor layer and, if appropriate, a layer that is arranged thereunder having A/D (analogue-to-digital) converters. Current CT devices are often cooled by a large flow of cooling air. All components of the CT device are connected to a common compressed air duct and thus receive the same air flow, which is only statically adapted through different ventilation holes. The component having the greatest cooling requirement specifies the amount of air to be provided. By connecting to a cooling water line, a temperature of the cooling air can also be controlled, for example via a heat exchanger, and a constant cooling capacity can be achieved accordingly.

The CT device, in particular a detector of the CT device, comprises a plurality of electronic components, for example semiconductor sensors. The electronic components, in particular the semiconductor sensors, are usually temperature-dependent. Thus, the output signals that are provided by the electronic components are also temperature-dependent. Frequently, the cooling capacity of present-day CT devices is controlled reactively, in particular in response to a request from a component of the CT device that more cooling, in particular a temperature reduction, is required. Thus, the adjustment of the cooling always takes place only at the moment when the leading component of the CT device is too warm, in particular a predefined maximum temperature has been reached or exceeded, and requests more cooling air. This form of cooling capacity control is disadvantageously retrospective and always has a control hysteresis.

### SUMMARY

It is therefore an object of one or more embodiments of the present invention to enable an improved temperature control of at least one component of an X-ray device to avoid thermal overshoot and/or undershoot.

At least this object is achieved in accordance with embodiments of the present invention by the subject matter of the independent claims. Advantageous embodiments having expedient developments are the subject matter of the subordinate claims.

Independent of the grammatical term usage, individuals with male, female or other gender identities are included within the term.

Embodiments of the present invention relate in a first aspect to a method for controlling the temperature of an X-ray device. In a first step, planning information is acquired comprising at least one operating parameter of at least one component of the X-ray device for planned operation of the X-ray device. In a further step, a planning temperature of the at least one component of the X-ray device is identified based on the at least one operating parameter. In a further step, the X-ray device is operated in accordance with the planning information. In this case, a temperature control unit of the X-ray device is controlled prior to and/or during the operation of the X-ray device, in particular at least prior to the operation, based on the planning temperature in such a manner that the temperature control unit controls the at least one component of the X-ray device to a predefined temperature or a predefined temperature range by providing a heating and/or cooling capacity.

The acquisition of the planning information can comprise receiving the planning information and/or acquiring the planning information on the basis of a user input. Receiving the planning information can comprise in particular acquiring and/or reading from a computer readable data storage device and/or receiving from a data storage unit, for example a database. Further, the planning information can be provided by a providing unit, for example, a medical device, in particular a CT device. Alternatively or additionally, the planning information can be acquired via an input unit for acquiring the user input, for example via a keyboard and/or a pointing device.

The planning information can comprise at least one operating parameter for at least one component of the X-ray device for the planned, in particular future, operation of the X-ray device. The planned operation of the X-ray device can denote an operation of the X-ray device that has not yet begun, in particular in the future. In particular, the planning information can comprise a plurality of operating parameters, in particular a plurality of different operating parameters and/or a plurality of values for one or a plurality of operating parameters, of at least one component, in particular a plurality of components, of the X-ray device. For example, the planning information can comprise a plurality of values for at least one operating parameter of the at least one component of the X-ray device, which predetermine a chronology, in particular a temporal sequence, of an operating parameter configuration of the at least one component of the X-ray device. Alternatively or additionally, the planning information can comprise at least one operating parameter for a plurality of components of the X-ray device. The at least one component of the X-ray device can, for example, comprise an X-ray source and/or an X-ray detector and/or a collimator wall and/or a motor of the X-ray device.

Advantageously, a planning temperature of the at least one component, in particular a respective planning temperature for each of the plurality of components, of the X-ray device can be identified based on the at least one operating parameter, in particular based on the plurality of operating parameters. The identification of the planning temperature can, for example, comprise a simulation and/or a calculation based on the at least one operating parameter and/or can be based on historical temperature values of the at least one component of the X-ray device and/or on, in particular, measured and/or simulated temperature values of a comparable at least one component of the X-ray device or of another X-ray device for the at least one operating parameter, for example via a look-up table. The planning temperature can advantageously denote a temperature value to be expected for the at least one component during the planned, in particular future, operation of the X-ray device, in particular without taking into account a heating and/or cooling capacity that can be provided by the temperature control unit.

The operation of the X-ray device in accordance with the planning information can comprise operating the at least one component of the X-ray device in accordance with the at least one operating parameter. Advantageously, the operation of the X-ray device in accordance with the planning information can be performed temporally after the acquisition of the planning information and the identification of the planning temperature. In particular, the X-ray device can be in a standby mode prior to the start of operation. The operation of the X-ray device in accordance with the planning information can, for example, comprise a recording operation and/or a positioning operation and/or a calibration operation of the X-ray device.

The temperature control unit can comprise at least one cooling element, for example a heat exchanger, and/or at least one heating element, for example a heating wire. The at least one cooling element and/or the at least one heating element can be arranged on the at least one component of the X-ray device that is to be temperature-controlled or can be integrated into the at least one component of the X-ray device that is to be temperature-controlled. Alternatively or additionally, the at least one cooling element and/or the at least one heating element can be arranged at a distance from the at least one component of the X-ray device that is to be temperature-controlled, wherein a heat transfer takes place between the at least one cooling element and/or the at least one heating element and the at least one component of the X-ray device via a heat-conducting medium, for example a fluid.

Advantageously, the temperature control unit of the X-ray device can be controlled prior to the operation of the X-ray device based on the planning temperature in such a manner that the temperature control unit controls the at least one component of the X-ray device to the predefined temperature or the predefined temperature range by providing the heating and/or cooling capacity. Alternatively or in addition, the temperature control unit of the X-ray device can be controlled during the operation of the X-ray device based on the planning temperature in such a manner that the temperature control unit controls the at least one component of the X-ray device to the predefined temperature or the predefined temperature range by providing the heating and/or cooling capacity.

The controlling of the temperature control unit based on the planning temperature can comprise controlling the heating element and/or the cooling element based on the planning temperature, in particular at least partially simultaneously or successively. The predefined temperature can denote a target temperature, in particular a target temperature value, for the at least one component of the X-ray device during the planned operation. The predefined temperature range can denote a target temperature interval for the at least one component of the X-ray device during the planned operation. Advantageously, the temperature control unit, in particular the at least one cooling element, can be designed so as to dissipate a cooling capacity, in particular a first defined amount of heat per unit of time, from the at least one component of the X-ray device. Alternatively or additionally, the temperature control unit, in particular the at least one heating element, can be designed so as to provide a heating capacity, in particular a further defined amount of heat per unit of time, to the at least one component of the X-ray device. Advantageously, the temperature control unit of the X-ray device can be controlled prior to and/or during the operation of the X-ray device based on the planning temperature in such a manner, for example via a processing unit, that the temperature control unit, in particular the at least one cooling element and/or the at least one heating element, can control, in particular cool or heat, the at least one component of the X-ray device to a predefined temperature or a predefined temperature range by providing a heating and/or cooling capacity.

The proposed method advantageously enables a pre-emptive control of the temperature control unit, in particular of the heating and/or cooling capacity to be provided, in order to avoid thermal overshoot and/or undershoot. Furthermore, via the proposed method, the at least one, in particular temperature-dependent, component of the X-ray device, for example a sensor and/or an electronic assembly, can be temperature controlled in a more uniform manner, in particular cooled and/or heated, and thus more uniform output signals can be provided by the at least one component. Furthermore, thermal fluctuations can be reduced and temperature cycles for the at least one component, in particular the plurality of components, for example assemblies and/or connections, of the X-ray device can be reduced, which can have a positive effect on a service life of the at least one component. A further advantage is that there is a harmonization of thermal states of the at least one component of the X-ray device during a tuning and during a measuring operation, in particular a patient operation. This can lead to an improved matching of tuning tables for later operating states of the X-ray device, for example during a clinical use of the X-ray device. Furthermore, via the proposed improved temperature control of the at least one component of the X-ray device, waiting times can be shortened by overheating the at least one component, in particular a post-cooling phase can be omitted or shortened.

In a further advantageous embodiment of the proposed method, the X-ray device can comprise an X-ray source and an X-ray detector. In this case, the planning information comprises at least one operating parameter of the X-ray source and/or the X-ray detector. Advantageously, the planned operation of the X-ray device comprises emitting X-rays via the X-ray source so as to illuminate the X-ray detector.

Advantageously, the X-ray device can comprise an X-ray source and an X-ray detector, for example a flat detector or a line detector, in particular a multi-line detector. In this case, the X-ray source can be designed so as to emit X-rays so as to illuminate an X-ray-sensitive surface of the X-ray detector. The X-ray detector can be designed for, in particular photon-counting, detection of the X-rays that are impinging on its X-ray-sensitive surface, in particular after fluoroscopy of an examination object to be imaged. Advantageously, the planning information comprises at least one operating parameter, in particular a plurality of operating parameters, of the X-ray source and/or at least one operating parameter, in particular a plurality of operating parameters, of the X-ray detector.

Advantageously, the planned operation of the X-ray device comprises emitting X-rays so as to illuminate the X-ray-sensitive surface of the X-ray detector, in particular in accordance with the at least one operating parameter of the X-ray source and/or the X-ray detector. In X-ray devices, most of the power is supplied by the X-ray source, in particular an X-ray tube. On the basis of the at least one operating parameter of the X-ray source, it is possible to identify points in time, a power quantity and/or a duration of action of a tube power of the X-ray tube prior to operating the X-ray device. Furthermore, an expected focal path temperature can be identified on the basis of the at least one operating parameter, for example via a tube load computer. Based on the focal path temperature, an expected waste heat and the planning temperature of the X-ray source can be identified. On this basis, a required cooling capacity for controlling the temperature of the X-ray source can be determined prior to operating the X-ray device. Advantageously, the temperature control unit can be controlled prior to and/or during the operation of the X-ray device based on the planning temperature in such a manner that a temperature increase of the X-ray source can be avoided and thermal overshoots can be significantly reduced.

In a further advantageous embodiment of the proposed method, the X-ray device can comprise an X-ray source and an X-ray detector. In this case, the planning information can comprise at least one operating parameter of the X-ray source and/or the X-ray detector. Furthermore, the X-ray source and the X-ray detector can be mounted in a movable, in particular rotatable, manner in a defined arrangement. In this case, the at least one operating parameter of the X-ray source and/or the X-ray detector comprises positioning information and/or movement information and/or information regarding a trajectory of the defined arrangement. Advantageously, furthermore a positioning-induced cooling capacity and/or heating capacity for the at least one component of the X-ray device is identified based on the positioning information and/or the movement information and/or the information regarding the trajectory of the defined arrangement. In this case, the temperature control unit can additionally be controlled based on the positioning-induced cooling capacity and/or heating capacity.

Advantageously, the X-ray source and the X-ray detector are arranged in a defined arrangement with respect to one another, for example on a common C-arm and/or a gantry. Furthermore, the defined arrangement of the X-ray source and the X-ray detector is mounted in a movable, in particular rotatable and/or translational manner. For example, the defined arrangement of the X-ray source and the X-ray detector can be arranged on a movable part of the X-ray device, for example a rotor of a gantry, in particular integrated into the rotor. The gantry can comprise an annular structure, in particular a stator, and the rotor. The rotor can be mounted in a rotatable manner about a rotation axis, in particular with respect to the stator. In this case, an examination object to be imaged, for example a patient and/or a phantom, can be arranged within an opening of the gantry, in particular between the X-ray source and the X-ray detector, for X-ray fluoroscopy. In this case, X-ray fluoroscopy refers to emitting X-rays via the X-ray source and detecting the X-rays after an interaction with the object to be examined via the X-ray detector.

Advantageously, the at least one operating parameter of the X-ray source and/or the X-ray detector comprises positioning information and/or movement information and/or a trajectory of the defined arrangement. The positioning information can describe a spatial position and/or orientation and/or pose of the X-ray source and/or the X-ray detector, in particular the defined arrangement of the X-ray source and the X-ray detector. The movement information can describe a movement direction and/or movement speed and/or acceleration of the X-ray source and/or the X-ray detector, in particular the defined arrangement of the X-ray source and the X-ray detector. The information regarding the trajectory of the defined arrangement can describe a spatial path of positioning of the defined arrangement, which is to be assumed by the X-ray source and/or the X-ray detector, in particular a defined arrangement, at different points in time, for example in temporal sequence, during the planned operation of the X-ray device. Thus, the operating parameter, in particular the positioning information and/or the movement information and/or the information regarding the trajectory, can characterize a rotational state and/or a change in the rotational state of the defined arrangement of the X-ray source and the X-ray detector, for example a gantry of a CT device.

Advantageously, a positioning-induced cooling capacity and/or heating capacity for the at least one component of the X-ray device is identified based on the positioning information and/or the movement information and/or the information regarding the trajectory of the defined arrangement. For example, the positioning of the X-ray source and/or the X-ray detector relative to at least one further component of the X-ray device and/or an object results in a positioning-induced heat transfer of the further component of the X-ray device and/or the object with the X-ray source and/or the X-ray detector. For example, the further component and/or the object can impede a heat transfer of the X-ray source and/or the X-ray detector with the temperature control unit. Alternatively or additionally, the further component and/or the object can transfer an amount of heat to the X-ray source and/or the X-ray detector or vice versa. Advantageously, the positioning-induced cooling capacity and/or heating capacity provided in this manner for the at least one component of the X-ray device can be identified based on the positioning information. In an analogous manner, the positioning of the X-ray source and/or the X-ray detector, in particular the defined arrangement, along the trajectory can lead to a positioning-induced cooling capacity and/or heating capacity for the at least one component of the X-ray device. Advantageously, the positioning-induced cooling capacity and/or heating capacity for the at least one component of the X-ray device can be identified based on the information regarding the trajectory of the defined arrangement.

Furthermore, the movement of the X-ray source and/or the X-ray detector, in particular the defined arrangement of the X-ray source and the X-ray detector, results in a movement-induced cooling capacity and/or heating capacity. For example, the movement of the defined arrangement relative to an air flow, for example an ambient air, can provide a cooling capacity to the X-ray source and/or the X-ray detector. In this case, the movement-induced cooling capacity can be dependent on a movement direction and/or a movement speed and/or an acceleration of the defined arrangement. For example, the cooling capacity in the case of a rapid movement, in particular a rapid rotation, of the defined arrangement can be higher than in the case of a comparatively slower movement, in particular a slow rotation, or in a static state of the defined arrangement. Alternatively or additionally, the movement of the X-ray source and/or the X-ray detector, in particular the defined arrangement, results in a movement-induced heating capacity, for example due to resistance forces counteracting the movement, in particular friction. Advantageously, the positioning-induced, in particular movement-induced, cooling capacity and/or heating capacity for the at least one component of the X-ray device can be identified based on the movement information.

The identification of the positioning-induced cooling capacity and/or heating capacity can comprise, for example, a simulation and/or calculation, in particular based on a look-up table, based on the position information and/or the movement information and/or the information regarding the trajectory of the defined arrangement and/or based on historical data about a positioning-induced cooling capacity and/or heating capacity for the at least one component of the X-ray device.

Advantageously, the temperature control unit can additionally be controlled based on the positioning-induced cooling capacity and/or heating capacity, for example via the processing unit. In particular, the positioning-induced cooling capacity and/or heating capacity can be taken into account when controlling the temperature control unit to control the temperature of the at least one component of the X-ray device to the predefined temperature and/or the predefined temperature range. The operation of the X-ray device in accordance with the operating parameter, comprising the positioning information and/or the movement information and/or the information regarding the trajectory of the defined arrangement, can comprise positioning and/or moving the defined arrangement, in particular with or without emitting X-rays, via the X-ray source for illuminating the X-ray detector.

In a further advantageous embodiment of the proposed method, the at least one operating parameter of the X-ray source can comprise a tube voltage and/or a tube power and/or a heating parameter and/or operating duration of the X-ray source. Alternatively or additionally, the at least one operating parameter of the X-ray detector can comprise a heating parameter of the X-ray detector.

The proposed embodiment can advantageously enable precise identification of the at least one planning temperature of the X-ray source and/or the X-ray detector based on the at least one operating parameter.

In a further advantageous embodiment of the proposed method, identifying the planning temperature can comprise a simulation of controlling the temperature of the at least one component of the X-ray device based on the at least one operating parameter.

Advantageously, identifying the planning temperature can comprise a simulation of controlling the temperature, in particular an auto-temperature control, of the at least one component of the X-ray device during the planned operation of the X-ray device based on the at least one operating parameter. The simulation can, for example, comprise a simulation based on a physical model of the at least one component and/or a simulation based on computational fluid dynamics (CFD). Advantageously, the planning temperature can be identified, in particular determined, based on the simulation of the temperature control of the at least one component of the X-ray device based on the at least one operating parameter.

For example, the X-ray device, in particular the at least one component of the X-ray device, can be provided as a digital twin, in particular a virtual representation, for example in software and/or a computer program product. A temporal thermal development of the at least one component, in particular the plurality of components, of the X-ray device can additionally be simulated via the digital twin. Based on this simulation, optimal control, in particular regulation, of the temperature control unit for providing the heating and/or cooling capacity can then take place. This optimization can also be performed by a learning algorithm, in particular an artificial intelligence, wherein respective conditions at the individual installation location of the X-ray device can be taken into account. Advantageously, the simulation can take into account technical limit values of the at least one component, for example a maximum temperature, as a boundary condition for the optimization. In this way, for example, overheating, in particular tube overheating at the X-ray source of the X-ray device, can be prevented.

In a further advantageous embodiment of the proposed method, the temperature control unit can comprise at least one cooling element and/or at least one heating element. In this case, the at least one cooling element is designed so as to dissipate a first defined amount of heat from the at least one component as cooling capacity. The at least one heating element can be designed so as to provide a further defined amount of heat to the at least one component as heating capacity. In this case, controlling the temperature of the at least one component of the X-ray device comprises dissipating a first defined amount of heat from the at least one component via the at least one cooling element and/or providing a further defined amount of heat to the at least one component via the at least one heating element.

The at least one cooling element can, for example, comprise a fan and/or a heat exchanger and/or an electrothermal converter. The at least one cooling element can be designed so as to dissipate the first defined amount of heat, in particular a first defined amount of heat per unit of time, from the at least one component of the X-ray device. The temperature control unit can, for example, comprise a cooling element that is designed so as to dissipate the first defined amount of heat, in particular the first defined amount of heat per unit of time, from a plurality of components of the X-ray device as cooling capacity. Alternatively, the temperature control unit can comprise a plurality of cooling elements, each of which is designed so as to dissipate a first defined amount of heat from one of the plurality of components of the X-ray device as cooling capacity. In particular, the temperature control unit can in each case comprise at least one cooling element for each of the plurality of components of the X-ray device.

The at least one, in particular optical and/or electrical and/or electromagnetic and/or chemical and/or mechanical, heating element can, for example, comprise a heating wire and/or a light source, in particular an infrared light source. The at least one heating element can be designed so as to dissipate the further defined amount of heat, in particular a further defined amount of heat per unit of time, to the at least one component of the X-ray device as heating capacity. The temperature control unit can, for example, comprise a heating element that is designed so as to provide the further defined amount of heat, in particular the further defined amount of heat per unit of time, to a plurality of components of the X-ray device as heating capacity. Alternatively, the temperature control unit can comprise a plurality of heating elements, each of which is designed so as to provide the further defined amount of heat to one of the plurality of components of the X-ray device as heating capacity. In particular, the temperature control unit can in each case comprise at least one heating element for each of the plurality of components of the X-ray device.

Advantageously, controlling the temperature of the at least one component of the X-ray device comprises dissipating a first defined amount of heat from the at least one component via the at least one cooling element and/or providing a further defined amount of heat to the at least one component of the X-ray device via the at least one heating element. The dissipation of the first defined amount of heat and the provision of the further defined amount of heat can take place at least partially one after the other or simultaneously.

For example, the cooling element can be designed so as to adjust the cooling capacity in a range from one to several minutes, while the heating element can be designed so as to adjust the heating capacity in a range from one to several seconds. In this case, the cooling capacity that is provided by the cooling element can be used for rough temperature control of the at least one component of the X-ray device, and the heating capacity that is provided by the heating element can be used for fine adjustment of the temperature control of the at least one component of the X-ray device.

In this manner, thermal overshoot and/or undershoot of the temperature of the at least one component of the X-ray device can advantageously be reduced or avoided.

In a further advantageous embodiment of the proposed method, the temperature control unit can comprise a fluid providing unit. In this case, controlling the temperature of the at least one component of the X-ray device comprises providing a fluid via the fluid providing unit.

The fluid providing unit can be designed so as to provide the fluid, in particular directly to the at least one component of the X-ray device or indirectly, for example to a fluid channel adjoining the at least one component of the X-ray device. The fluid providing unit can, for example, comprise a pump and/or a fan and/or a nozzle. The fluid can comprise, for example, a liquid, in particular water and/or oil, and/or a gas and/or a gas mixture, in particular air. The at least one component of the X-ray device can advantageously be in thermally conductive contact with the fluid that is provided. In this case, the fluid can be controlled to a further predefined temperature and/or a further predefined temperature range via the temperature control unit, in particular the cooling element and/or the heating element, prior to being provided. In this manner, the fluid that is temperature-controlled to the further predefined temperature and/or the further predefined temperature range can come into thermally conductive contact with the at least one component of the X-ray device. In this case, heat can be transferred between the fluid and the at least one component of the X-ray device.

The proposed embodiment can enable particularly efficient temperature control of the at least one component of the X-ray device.

In a further advantageous embodiment of the proposed method, a current temperature of the at least one component can be acquired via a sensor. In this case, the temperature control unit of the X-ray device can be additionally controlled based on the current temperature of the at least one component of the X-ray device so as to provide the heating and/or cooling capacity.

The sensor can comprise a temperature sensor, in particular an optical and/or electromagnetic and/or mechanical and/or chemical temperature sensor. Advantageously, the temperature sensor can be designed so as to detect the current temperature of the at least one component of the X-ray device, in particular prior to operating the X-ray device. In particular, the temperature sensor can be designed so as to provide a signal in dependence upon the acquired current temperature of the at least one component of the X-ray device. The temperature sensor can advantageously be arranged on the at least one component of the X-ray device or at least partially, in particular completely, integrated into the at least one component of the X-ray device.

Advantageously, the temperature control unit of the X-ray device can be additionally controlled based on the current temperature of the at least one component of the X-ray device so as to provide the heating and/or cooling capacity. In particular, a temperature difference between the predefined temperature or the predefined temperature range and the current temperature can be identified based on the current temperature of the at least one component, in particular prior to operation of the X-ray device. In this case, the temperature control unit can be designed so as to adapt the heating and/or cooling capacity to be provided in dependence upon the identified temperature difference. For example, the at least one component of the X-ray device can have been heated to the current temperature by a preprocedural operation, in particular prior to the start of the method. As a result, the temperature difference between the predefined temperature or the predefined temperature range and the current temperature can be smaller compared to a preprocedural idle state of the X-ray device. Advantageously, this difference in the temperature difference can be taken into account via the proposed embodiment when controlling the temperature of the at least one component of the X-ray device via the temperature control unit.

The proposed embodiment can advantageously enable particularly precise temperature control of the at least one component of the X-ray device.

In a further advantageous embodiment of the proposed method, the planning information for a plurality of components of the X-ray device can in each case comprise at least one operating parameter for the planned operation of the X-ray device. In this case, a respective planning temperature for each of the plurality of components of the X-ray device can be identified based on the operating parameters, Moreover, the temperature control unit can control the plurality of components of the X-ray device to a predefined temperature or a predefined temperature range in each case by providing the heating and/or cooling capacity.

Advantageously, the planning information for a plurality of components, in particular a plurality of different components, of the X-ray device in each case can comprise at least one, in particular a plurality, of operating parameters for the planned operation of the X-ray device. Advantageously, a respective planning temperature for each of the plurality of components of the X-ray device can be identified based on the respective operating parameters or the plurality of operating parameters altogether. The plurality of planning temperatures for the plurality of components of the X-ray device can be at least partially, in particular completely, different or the same.

Advantageously, the temperature control unit can control the temperature of the plurality of components of the X-ray device in each case to the predefined temperature or to a respective predefined temperature range by providing the heating and/or cooling capacity. The plurality of components of the X-ray device can have at least partially, in particular completely, different or the same predefined temperatures or predefined temperature ranges. Advantageously, the temperature of the plurality of components of the X-ray device can be controlled hierarchically via the temperature control unit. In this case, at least one of the plurality of components of the X-ray device, in particular a selected one of the plurality of components of the X-ray device, can make a request to the temperature control unit for providing the heating and/or cooling capacity based on its predefined temperature and/or its predefined temperature range. The temperature control unit can meet this request by providing the heating and/or cooling capacity prioritized over the rest of the plurality of components of the X-ray device.

The proposed embodiment can enable improved coordination of the temperature control of the plurality of components of the X-ray device, in particular taking into account individual technical limit values of the respective components.

Embodiments of the present invention relate in a second aspect to an X-ray device, in particular a medical X-ray device, comprising a processing unit, an X-ray source, an X-ray detector and a temperature control unit. The X-ray source is designed so as to emit X-rays. The X-ray detector is designed so as to detect the X-rays. The temperature control unit is designed so as to provide a heating and/or cooling capacity to at least one component of the X-ray device. The processing unit is designed so as to acquire planning information comprising at least one operating parameter of the at least one component of the X-ray device for planned operation of the X-ray device. The processing unit is furthermore designed so as to identify a planning temperature of the at least one component of the X-ray device based on the at least one operating parameter. The processing unit controls the X-ray device so as to operate in an operating state in accordance with the at least one operating parameter. In the operating state the processing unit controls the temperature control unit based on the planning temperature in such a manner that the temperature control unit controls the at least one component of the X-ray device to a predefined temperature or a predefined temperature range by providing a heating and/or cooling capacity.

The advantages of the proposed X-ray device correspond essentially to the advantages of the proposed method for controlling the temperature of an X-ray device. In this case, mentioned features, advantages or alternative embodiments can likewise also be transferred to the other claimed subjects and vice versa.

Advantageously, the X-ray device can be designed as a computer tomography device (CT device) and/or a C-arm X-ray device and/or an O-arm X-ray device.

In a further advantageous embodiment of the proposed X-ray device, the X-ray source and the X-ray detector are mounted in a movable manner in a defined arrangement. Furthermore, the planning information can comprise at least one operating parameter of the X-ray source and/or the X-ray detector. Moreover, the at least one operating parameter of the X-ray source and/or the X-ray detector can comprise position information and/or movement information and/or information regarding a trajectory of the defined arrangement. The processing unit can be designed so as to identify a positioning-induced cooling capacity and/or heating capacity for the at least one component of the X-ray device based on the positioning information and/or the movement information and/or the information regarding the trajectory and to control the temperature control unit in the operating state additionally based on the positioning-induced cooling capacity and/or heating capacity.

In a further advantageous embodiment of the proposed X-ray device, the temperature control unit can comprise a fluid providing unit, which is designed so as to provide a fluid. The temperature control unit can furthermore be designed so as to provide the heating and/or cooling capacity to the at least one component of the X-ray device via a heat transfer between the at least one component and the fluid.

In a further advantageous embodiment of the proposed X-ray device, the temperature control unit can comprise a cooling element and/or a heating element. In this case, the cooling element can be designed so as to dissipate a first defined amount of heat from the at least one component as cooling capacity. The heating element can be designed so as to provide a further defined amount of heat to the at least one component as heating capacity. The temperature control unit can be designed so as to control the temperature of at least one component of the X-ray device by dissipating a first defined amount of heat from the at least one component via the cooling element and/or by providing a further defined amount of heat to the at least one component via the heating element.

In a further advantageous embodiment of the proposed X-ray device, the X-ray device can furthermore comprise a sensor that is designed so as to detect a current temperature of the at least one component of the X-ray device. The processing unit can advantageously be designed so as to control the temperature control unit in the operating state additionally based on the current temperature of the at least one component.

Embodiments of the present invention relate in a third aspect to a computer program product having a computer program that can be loaded directly into a storage device of a processing unit, having program sections in order to perform all the steps of a proposed method for controlling the temperature of an X-ray device if the program sections are performed by the processing unit.

In this case, the computer program product can comprise software having a source code that still has to be compiled and bound or only has to be interpreted, or an executable software code that only has to be loaded into the processing unit for execution. Due to the computer program product, the method for controlling the temperature of an X-ray device can be implemented in a fast, identically repeatable and robust manner via a processing unit. The computer program product is configured in such a manner that it can perform the method steps, in accordance with embodiments of the present invention, via the processing unit.

The computer program product is, for example, stored on a non-transitory computer-readable storage medium or stored on a network or server, from where it can be loaded into the processor of a processing unit, which can be directly connected to the processing unit or can be designed as part of the processing unit. Furthermore, control information of the computer program product can be stored on an electronically readable data carrier. The control information of the electronically readable data carrier can be designed in such a manner that, when the data carrier is used in a processing unit, it implements a method, in accordance with embodiments of the present invention. Examples of electronically readable data carriers are a DVD, a magnetic tape or a USB stick, on which electronically readable control information, in particular software, is stored. When this control information is read from the data carrier and stored in a processing unit, all embodiments in accordance with the present invention of the methods described above can be implemented.

A largely software-based realization has the advantage that processing units that are already previously used can be retrofitted in a simple manner by a software update in order to function in the manner in accordance with embodiments of the present invention. Such a computer program product in addition to the computer program where necessary can comprise additional components such as for example a documentation and/or additional components, and also hardware components such as for example hardware keys (dongles etc.) in order to use the software.

### BRIEF DESCRIPTION OF THE DRAWINGS

Exemplary embodiments of the present invention are illustrated in the drawings and are further described below. The same reference characters are used for identical features in different figures. In the drawings:

FIGS. 1, 2 and 3 show schematic illustrations of various advantageous embodiments of a proposed method for controlling the temperature of an X-ray device,

FIG. 4 shows a schematic illustration of an advantageous embodiment of a proposed X-ray device.

### DETAILED DESCRIPTION

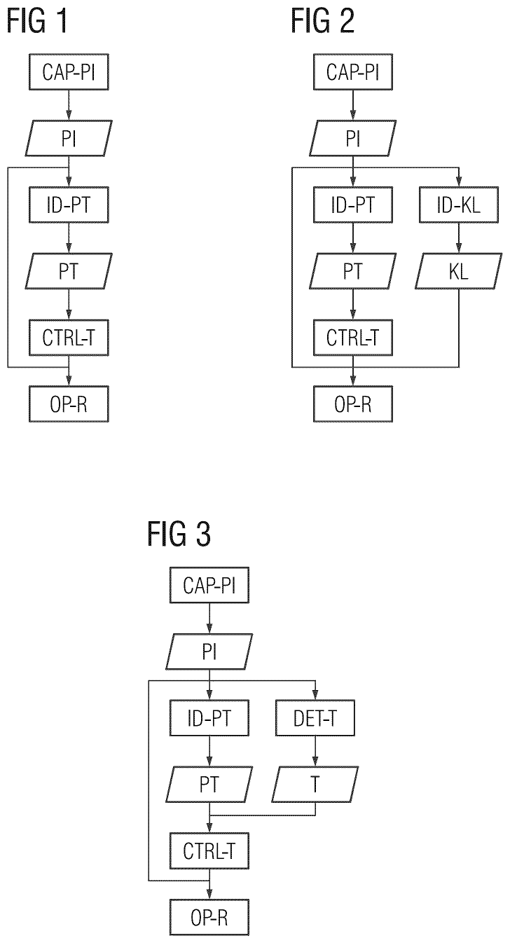

FIG. 1 illustrates a schematic illustration of an advantageous embodiment of a proposed method for controlling the temperature of an X-ray device. In a first step, planning information PI can be acquired comprising at least one operating parameter of at least one component of the X-ray device for planned operation of the X-ray device CAP-PI. In a further step, a planning temperature PT of the at least one component of the X-ray device can be identified ID-PT based on the at least one operating parameter. Advantageously, identifying (ID-PT) the planning temperature (PT) can comprise a simulation of controlling the temperature of the at least one component of the X-ray device based on the at least one operating parameter. In a further step, the X-ray device can be operated OP-R in accordance with the planning information PI. In this case, a temperature control unit of the X-ray device can be controlled CTRL-T prior to and/or during the operation of the X-ray device, in particular at least prior to the operation of the X-ray device, based on the planning temperature PT in such a manner that the temperature control unit controls the at least one component of the X-ray device to a predefined temperature or a predefined temperature range by providing a heating and/or cooling capacity.

Advantageously, the X-ray device can comprise an X-ray source and an X-ray detector. In this case, the planning information PI can comprise at least one operating parameter of the X-ray source and/or the X-ray detector. Furthermore, the planned operation of the X-ray device can comprise emitting X-rays via the X-ray source so as to illuminate the X-ray detector. In this case, the at least one operating parameter of the X-ray source can comprise a tube voltage and/or a tube power and/or a heating parameter and/or operating duration of the X-ray source. Alternatively or additionally, the at least one operating parameter of the X-ray detector can comprise a heating parameter of the X-ray detector.

Advantageously, the temperature control unit can comprise a cooling element and/or a heating element. In this case, the cooling element can be designed so as to dissipate a first defined amount of heat from the at least one component as cooling capacity. Furthermore, the heating element can be designed so as to provide a further defined amount of heat to the at least one component as heating capacity. In this case, controlling the temperature of the at least one component of the X-ray device comprises dissipating a first defined amount of heat from the at least one component via the cooling element and/or providing a further defined amount of heat to the at least one component via the heating element. In particular, the temperature control unit can comprise a fluid providing unit. In this case, controlling the temperature of the at least one component of the X-ray device can comprise providing a fluid via the fluid providing unit. In this case, heat can be transferred between the at least one component and the fluid.

Advantageously, the planning information for a plurality of components of the X-ray device can comprise in each case at least one operating parameter for the planned operation of the X-ray device. In this case, a respective planning temperature for each of the plurality of components of the X-ray device can be identified ID-PT based on the operating parameters. Furthermore, the temperature control unit can control the plurality of components of the X-ray device to a predefined temperature or a predefined temperature range in each case by providing the heating and/or cooling capacity.

FIG. 2 illustrates a schematic illustration of a further advantageous embodiment of a proposed method for controlling the temperature of an X-ray device. Moreover, the X-ray source and the X-ray detector are mounted in a movable, in particular rotatable, manner in a defined arrangement. In this case, the at least one operating parameter of the X-ray source and/or the X-ray detector comprises positioning information and/or movement information and/or information regarding a trajectory of the defined arrangement.

Furthermore, a positioning-induced cooling capacity and/or heating capacity KL for the at least one component of the X-ray device is identified ID-KL based on the positioning information and/or the movement information and/or the information regarding the trajectory. In this case, the temperature control unit can additionally be controlled CTRL-T based on the positioning-induced cooling capacity and/or heating capacity KL.

FIG. 3 illustrates a schematic illustration of a further advantageous embodiment of a proposed method for controlling the temperature of an X-ray device. In this case, a current temperature T of the at least one component can be acquired DET-T via a sensor. The temperature control unit of the X-ray device can advantageously be additionally controlled based on the current temperature of the at least one component of the X-ray device so as to provide the heating and/or cooling capacity.

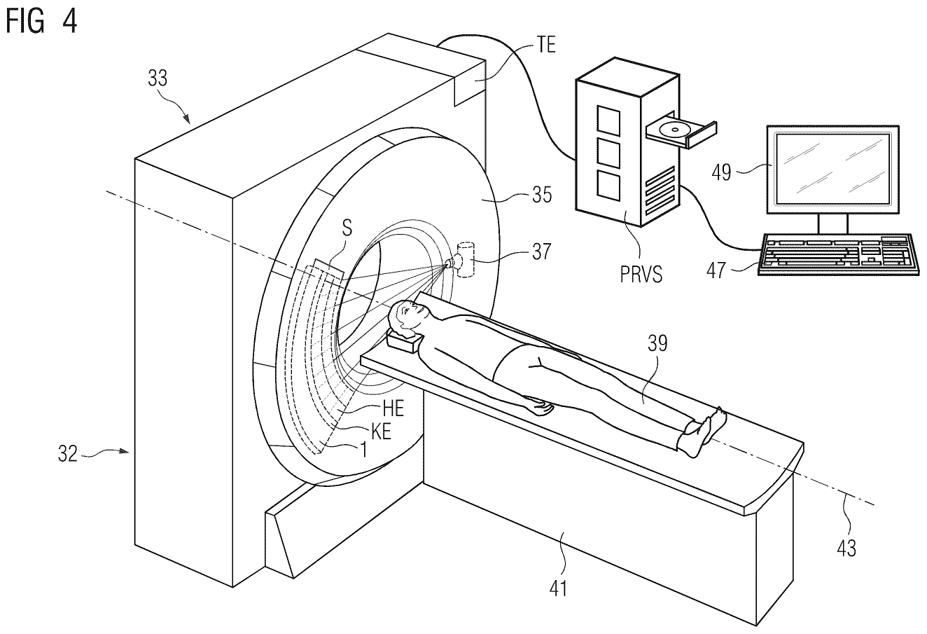

FIG. 4 illustrates a schematic illustration of an advantageous embodiment of a proposed X-ray device as a medical CT device 33. The CT device 33 can comprise the X-ray source 37, the X-ray detector 1 and a processing unit PRVS. In this case, the X-ray source 37 and the X-ray detector 1 can be arranged opposite one another. The X-ray source 37 can be designed so as to emit X-rays. In particular, the X-ray source 37 can be designed so as to expose the X-ray detector 1 to X-rays along an X-ray incidence direction. The X-ray detector 1 can comprise a direct-converting (semiconductor) X-ray detector layer. In this case, the X-ray detector layer can have, for example, CdTe, CdZnTe, CdTeSe, CdZnTeSe or CdMnTe as the semiconductor material. The X-ray detector layer can also comprise a layer with analog-to-digital converters, to which the X-ray detector layer is applied, wherein the A/D converter layer can be realized in one or more ASICs. The X-ray detector can be designed so as to detect the X-rays.

The CT system 33 moreover can comprise a gantry 32 having a rotor 35. The X-ray source 37 and the X-ray detector 1 can be arranged in a defined arrangement on the rotor 35, in particular integrated into the rotor 35 or fastened to the rotor 35. The rotor 35 can be mounted in a rotatable manner about a rotation axis 43. The examination object to be imaged 39 can be mounted on the patient mounting apparatus 41 and is movable along the axis of rotation 43 through the gantry 32. The processing unit PRVS can be used to control the CT device 33 and to calculate sectional images or volume images of the examination object 39. Advantageously, the processing unit PRVS can be designed so as to acquire CAP-PI the planning information PI comprising the at least one operating parameter of the at least one component of the CT device 33 for the planned operation of the CT device 33. The processing unit PRVS can furthermore be designed so as to identify ID-PT the planning temperature PT of the at least one component of the CT device 33 based on the at least one operating parameter.

The CT device 33 can further comprise the temperature control unit TE. The temperature control unit TE can be designed so as to provide a heating and/or cooling capacity to at least one component of the CT device 33. Advantageously, the processing unit PRVS can control the CT device 33 in an operating state in accordance with the at least one operating parameter for the operation OP-R. In the operating state, the processing unit PRVS can control CTRL-T the temperature control unit TE based on the planning temperature PT in such a manner that the temperature control unit TE controls the at least one component of the X-ray device to a predefined temperature or a predefined temperature range by providing a heating and/or cooling capacity.

An input facility 47, for example a keyboard, and an output apparatus 49, for example a screen and/or display, can be connected to the processing unit PRVS, in particular coupled in terms of signal technology. The input facility 47 can advantageously be integrated into the output apparatus 49, for example in the case of an input display, in particular a resistive and/or capacitive input display.

Advantageously, the planning information PI can comprise at least one operating parameter of the X-ray source 37 and/or the X-ray detector 1. In this case, the at least one operating parameter of the X-ray source 37 and/or the X-ray detector 1 can comprise positioning information and/or movement information and/or information regarding a trajectory of the defined arrangement. Furthermore, the processing unit PRVS can be designed so as to identify ID-KL a positioning-induced cooling capacity and/or heating capacity KL for the at least one component of the CT device 33 based on the movement information and/or the information regarding the trajectory and to control CTRL-T the temperature control unit TE in the operating state additionally based on the positioning-induced cooling capacity and/or heating capacity.

Advantageously, the temperature control unit TE can further comprise a fluid providing unit (not shown here), which is designed so as to provide a fluid. In this case, the temperature control unit TE is designed so as to provide the heating and/or cooling power to the at least one component of the CT device 33 via a heat transfer between the at least one component and the fluid.

The temperature control unit TE can further comprise a cooling element KE and/or a heating element HE. The cooling element KE can be designed so as to dissipate a first defined amount of heat from the at least one component as cooling capacity. Furthermore, the heating element HE can be designed so as to provide a further defined amount of heat to the at least one component as heating capacity. The temperature control unit TE can be designed so as to control the temperature of the at least one component of the CT device 33 by dissipating a first defined amount of heat from the at least one component via the cooling element KE and/or by providing a further defined amount of heat to the at least one component via the heating element HE.

The CT device 33 can advantageously furthermore comprise a sensor S that is designed so as to acquire CAP-T a current temperature T of the at least one component of the CT device. In this case, the processing unit PRVS can furthermore be designed so as to control CTRL-T the temperature control unit TE in the operating state additionally based on the current temperature T of the at least one component.

The schematic representations that are included in the described figures do not depict any scale or proportions.

Finally, reference is again made to the fact that the method and illustrated apparatuses that are described above in detail are only exemplary embodiments that can be modified in various ways by the person skilled in the art without departing the scope of the present invention. Furthermore, the use of the indefinite article “a” or “an” does not rule out that the relevant features can also be provided multiple times. Likewise, the terms “unit” and “element” do not rule out that the relevant components are made of multiple interacting part components that where necessary can also be spatially distributed.

The term “based on” can be understood in the context of the present application in particular in the sense of the term “using”. In particular, a wording according to which a first feature is produced based on a second feature (alternatively: identified, determined, etc.) does not exclude that the first feature can further be produced based on a third feature (alternatively: identified, determined etc.).

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of example embodiments. As used herein, the term “and/or,” includes any and all combinations of one or more of the associated listed items. The phrase “at least one of” has the same meaning as “and/or”.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “on,” “connected,” “engaged,” “interfaced,” and “coupled.” Unless explicitly described as being “direct,” when a relationship between first and second elements is described in the disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” on, connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “example” is intended to refer to an example or illustration.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

It is noted that some example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed above. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing example embodiments. The present invention may, however, be embodied in many alternate forms and should not be construed as limited to only the embodiments set forth herein.

In addition, or alternative, to that discussed above, units and/or devices according to one or more example embodiments may be implemented using hardware, software, and/or a combination thereof. For example, hardware devices may be implemented using processing circuitry such as, but not limited to, a processor, Central Processing Unit (CPU), a controller, an arithmetic logic unit (ALU), a digital signal processor, a microcomputer, a field programmable gate array (FPGA), a System-on-Chip (SoC), a programmable logic unit, a microprocessor, or any other device capable of responding to and executing instructions in a defined manner. Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

In this application, including the definitions below, the term ‘module’ or the term ‘controller’ may be replaced with the term ‘circuit.’ The term ‘module’ may refer to, be part of, or include processor hardware (shared, dedicated, or group) that executes code and memory hardware (shared, dedicated, or group) that stores code executed by the processor hardware.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

Software may include a computer program, program code, instructions, or some combination thereof, for independently or collectively instructing or configuring a hardware device to operate as desired. The computer program and/or program code may include program or computer-readable instructions, software components, software modules, data files, data structures, and/or the like, capable of being implemented by one or more hardware devices, such as one or more of the hardware devices mentioned above. Examples of program code include both machine code produced by a compiler and higher level program code that is executed using an interpreter.

For example, when a hardware device is a computer processing device (e.g., a processor, Central Processing Unit (CPU), a controller, an arithmetic logic unit (ALU), a digital signal processor, a microcomputer, a microprocessor, etc.), the computer processing device may be configured to carry out program code by performing arithmetical, logical, and input/output operations, according to the program code. Once the program code is loaded into a computer processing device, the computer processing device may be programmed to perform the program code, thereby transforming the computer processing device into a special purpose computer processing device. In a more specific example, when the program code is loaded into a processor, the processor becomes programmed to perform the program code and operations corresponding thereto, thereby transforming the processor into a special purpose processor.

Software and/or data may be embodied permanently or temporarily in any type of machine, component, physical or virtual equipment, or computer storage medium or device, capable of providing instructions or data to, or being interpreted by, a hardware device. The software also may be distributed over network coupled computer systems so that the software is stored and executed in a distributed fashion. In particular, for example, software and data may be stored by one or more computer readable recording mediums, including the tangible or non-transitory computer-readable storage media discussed herein.

Even further, any of the disclosed methods may be embodied in the form of a program or software. The program or software may be stored on a non-transitory computer readable medium and is adapted to perform any one of the aforementioned methods when run on a computer device (a device including a processor). Thus, the non-transitory, tangible computer readable medium, is adapted to store information and is adapted to interact with a data processing facility or computer device to execute the program of any of the above mentioned embodiments and/or to perform the method of any of the above mentioned embodiments.

Example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order.

According to one or more example embodiments, computer processing devices may be described as including various functional units that perform various operations and/or functions to increase the clarity of the description. However, computer processing devices are not intended to be limited to these functional units. For example, in one or more example embodiments, the various operations and/or functions of the functional units may be performed by other ones of the functional units. Further, the computer processing devices may perform the operations and/or functions of the various functional units without sub-dividing the operations and/or functions of the computer processing units into these various functional units.

Units and/or devices according to one or more example embodiments may also include one or more storage devices. The one or more storage devices may be tangible or non-transitory computer-readable storage media, such as random access memory (RAM), read only memory (ROM), a permanent mass storage device (such as a disk drive), solid state (e.g., NAND flash) device, and/or any other like data storage mechanism capable of storing and recording data. The one or more storage devices may be configured to store computer programs, program code, instructions, or some combination thereof, for one or more operating systems and/or for implementing the example embodiments described herein. The computer programs, program code, instructions, or some combination thereof, may also be loaded from a separate computer readable storage medium into the one or more storage devices and/or one or more computer processing devices using a drive mechanism. Such separate computer readable storage medium may include a Universal Serial Bus (USB) flash drive, a memory stick, a Blu-ray/DVD/CD-ROM drive, a memory card, and/or other like computer readable storage media. The computer programs, program code, instructions, or some combination thereof, may be loaded into the one or more storage devices and/or the one or more computer processing devices from a remote data storage device via a network interface, rather than via a local computer readable storage medium. Additionally, the computer programs, program code, instructions, or some combination thereof, may be loaded into the one or more storage devices and/or the one or more processors from a remote computing system that is configured to transfer and/or distribute the computer programs, program code, instructions, or some combination thereof, over a network. The remote computing system may transfer and/or distribute the computer programs, program code, instructions, or some combination thereof, via a wired interface, an air interface, and/or any other like medium.

The one or more hardware devices, the one or more storage devices, and/or the computer programs, program code, instructions, or some combination thereof, may be specially designed and constructed for the purposes of the example embodiments, or they may be known devices that are altered and/or modified for the purposes of example embodiments.

A hardware device, such as a computer processing device, may run an operating system (OS) and one or more software applications that run on the OS. The computer processing device also may access, store, manipulate, process, and create data in response to execution of the software. For simplicity, one or more example embodiments may be exemplified as a computer processing device or processor; however, one skilled in the art will appreciate that a hardware device may include multiple processing elements or processors and multiple types of processing elements or processors. For example, a hardware device may include multiple processors or a processor and a controller. In addition, other processing configurations are possible, such as parallel processors.

The computer programs include processor-executable instructions that are stored on at least one non-transitory computer-readable medium (memory). The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc. As such, the one or more processors may be configured to execute the processor executable instructions.

The computer programs may include: (i) descriptive text to be parsed, such as HTML (hypertext markup language) or XML (extensible markup language), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C#, Objective-C, Haskell, Go, SQL, R, Lisp, Java®, Fortran, Perl, Pascal, Curl, OCaml, Javascript®, HTML5, Ada, ASP (active server pages), PHP, Scala, Eiffel, Smalltalk, Erlang, Ruby, Flash®, Visual Basic®, Lua, and Python®.

Further, at least one example embodiment relates to the non-transitory computer-readable storage medium including electronically readable control information (processor executable instructions) stored thereon, configured in such that when the storage medium is used in a controller of a device, at least one embodiment of the method may be carried out.

The computer readable medium or storage medium may be a built-in medium installed inside a computer device main body or a removable medium arranged so that it can be separated from the computer device main body. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

Although described with reference to specific examples and drawings, modifications, additions and substitutions of example embodiments may be variously made according to the description by those of ordinary skill in the art. For example, the described techniques may be performed in an order different with that of the methods described, and/or components such as the described system, architecture, devices, circuit, and the like, may be connected or combined to be different from the above-described methods, or results may be appropriately achieved by other components or equivalents.

What is claimed is:

1. A method for controlling a temperature of an X-ray device, the method comprising:
acquiring planning information for planned operation of the X-ray device, the planning information including at least one operating parameter of at least one component of the X-ray device;
identifying a planning temperature of the at least one component of the X-ray device based on the at least one operating parameter;
operating the X-ray device in accordance with the planning information; and
controlling, based on the planning temperature, a temperature control unit of the X-ray device at least one of prior to or during operation of the X-ray device such that the temperature control unit controls a temperature of the at least one component of the X-ray device to a defined temperature or a defined temperature range by providing at least one of a heating capacity or a cooling capacity.

2. The method as claimed in claim 1,
wherein the X-ray device includes an X-ray source and an X-ray detector,
wherein the planning information includes at least one operating parameter of at least one of the X-ray source or the X-ray detector, and
wherein the planned operation of the X-ray device includes emitting X-rays via the X-ray source to illuminate the X-ray detector.

3. The method as claimed in claim 1,
wherein the X-ray device includes an X-ray source and an X-ray detector,
wherein the planning information includes at least one operating parameter of at least one of the X-ray source or the X-ray detector,
wherein the X-ray source and the X-ray detector are mounted in a movable manner in a defined arrangement,
wherein the at least one operating parameter of at least one of the X-ray source or the X-ray detector includes at least one of positioning information, movement information or a trajectory of the defined arrangement,
wherein at least one of a positioning-induced cooling capacity or a positioning-induced heating capacity for the at least one component of the X-ray device is identified based on the at least one of the positioning information, the movement information or the trajectory, and
wherein the temperature control unit is additionally controlled based on the at least one of the positioning-induced cooling capacity or the positioning-induced heating capacity.

4. The method as claimed in claim 2, wherein at least one of
at least one operating parameter of the X-ray source includes at least one of a tube voltage, a tube power, a heating parameter, or an operating duration of the X-ray source, or
at least one operating parameter of the X-ray detector includes a heating parameter of the X-ray detector.

5. The method as claimed in claim 1, wherein identifying the planning temperature includes a simulation of controlling the temperature of the at least one component of the X-ray device based on the at least one operating parameter.

6. The method as claimed in claim 1,
wherein the temperature control unit includes at least one of a cooling element or a heating element,
wherein the cooling element is configured to dissipate a first defined amount of heat from the at least one component as the cooling capacity,
wherein the heating element is configured to provide a further defined amount of heat to the at least one component as the heating capacity, and
wherein controlling the temperature of the at least one component of the X-ray device includes at least one of dissipating the first defined amount of heat from the at least one component via the cooling element or providing the further defined amount of heat to the at least one component via the heating element.

7. The method as claimed in claim 1,
wherein the temperature control unit includes a fluid providing unit,
wherein controlling the temperature of the at least one component of the X-ray device includes providing a fluid via the fluid providing unit, and
wherein heat is transferred between the at least one component and the fluid.

8. The method as claimed in claim 1,
wherein a current temperature of the at least one component is acquired via a sensor, and
wherein the temperature control unit of the X-ray device is additionally controlled based on the current temperature of the at least one component of the X-ray device to provide the at least one of the heating capacity or cooling capacity.

9. The method as claimed in claim 1,
wherein planning information for a plurality of components of the X-ray device, in each case, includes at least one operating parameter for the planned operation of the X-ray device,
wherein a respective planning temperature for each of the plurality of components of the X-ray device is identified based on the operating parameters, and
wherein the temperature control unit controls the plurality of components of the X-ray device to the defined temperature or the defined temperature range by providing the at least one of the heating capacity or the cooling capacity.

10. An X-ray device comprising:
an X-ray source configured to emit X-rays;
an X-ray detector configured to detect the X-rays;
a temperature control unit configured to provide at least one of a heating capacity or a cooling capacity to at least one component of the X-ray device; and
a processing unit configured to
acquire planning information for planned operation of the X-ray device, the planning information including at least one operating parameter of the at least one component of the X-ray device,
identify a planning temperature of the at least one component of the X-ray device based on the at least one operating parameter, and
control the X-ray device to operate in an operating state in accordance with the at least one operating parameter, wherein
in the operating state, the processing unit is configured to control the temperature control unit based on the planning temperature such that the temperature control unit controls a temperature of the at least one component of the X-ray device to a defined temperature or a defined temperature range by providing the at least one of the heating capacity or the cooling capacity.

11. The X-ray device as claimed in claim 10,
wherein the X-ray source and the X-ray detector are mounted in a movable manner in a defined arrangement,
wherein the planning information includes at least one operating parameter of at least one of the X-ray source or the X-ray detector,
wherein the at least one operating parameter of the at least one of the X-ray source or the X-ray detector includes at least one of positioning information, movement information or information regarding a trajectory of the defined arrangement,
wherein the processing unit is configured to
identify at least one of a positioning-induced cooling capacity or a positioning-induced heating capacity for the at least one component of the X-ray device based on the at least one of the positioning information, the movement information or the information regarding the trajectory, and
control the temperature control unit in the operating state additionally based on the at least one of the positioning-induced cooling capacity or the positioning-induced heating capacity.

12. The X-ray device as claimed in claim 10,
wherein the temperature control unit includes a fluid providing unit, which is configured to provide a fluid, and
wherein the temperature control unit is configured to provide the at least one of the heating capacity or the cooling capacity to the at least one component of the X-ray device via a heat transfer between the at least one component and the fluid.

13. The X-ray device as claimed in claim 10,
wherein the temperature control unit includes at least one of a cooling element or a heating element,
wherein the cooling element is configured to dissipate a first defined amount of heat from the at least one component as the cooling capacity,
wherein the heating element is configured to provide a further defined amount of heat to the at least one component as the heating capacity, and
wherein the temperature control unit is configured to control the temperature of the at least one component of the X-ray device by at least one of dissipating the first defined amount of heat from the at least one component via the cooling element, or providing the further defined amount of heat to the at least one component via the heating element.

14. The X-ray device as claimed in claim 10, further comprising:
a sensor configured to acquire a current temperature of the at least one component of the X-ray device, and wherein
the processing unit is configured to control the temperature control unit in the operating state additionally based on the current temperature of the at least one component.

15. A non-transitory computer-readable storage medium storing computer-executable instructions that, when executed at a processing unit, cause the processing unit to perform the method of claim 1.

16. The method of claim 3, wherein the X-ray source and the X-ray detector are mounted in a rotatable manner in the defined arrangement.

17. The method as claimed in claim 3, wherein at least one of
at least one operating parameter of the X-ray source includes at least one of a tube voltage, a tube power, a heating parameter, or an operating duration of the X-ray source, or
at least one operating parameter of the X-ray detector includes a heating parameter of the X-ray detector.

18. The method as claimed in claim 3,
wherein the temperature control unit includes at least one of a cooling element or a heating element,
wherein the cooling element is configured to dissipate a first defined amount of heat from the at least one component as the cooling capacity,
wherein the heating element is configured to provide a further defined amount of heat to the at least one component as the heating capacity, and
wherein controlling the temperature of the at least one component of the X-ray device includes at least one of dissipating the first defined amount of heat from the at least one component via the cooling element or providing the further defined amount of heat to the at least one component via the heating element.

19. The method as claimed in claim 3,
wherein the temperature control unit includes a fluid providing unit,
wherein controlling the temperature of the at least one component of the X-ray device includes providing a fluid via the fluid providing unit, and
wherein heat is transferred between the at least one component and the fluid.

20. The method as claimed in claim 3,
wherein a current temperature of the at least one component is acquired via a sensor, and
wherein the temperature control unit of the X-ray device is additionally controlled based on the current temperature of the at least one component of the X-ray device to provide the at least one of the heating capacity or cooling capacity.
